(19) United States
(12) Patent Application Publication
(10) Pub. No.: US 2026/0211128 A1
(43) Pub. Date: Jul. 23, 2026

(54) RADIATION IMAGING APPARATUS AND RADIATION IMAGE PROCESSING METHOD

(71) Applicant: SHIMADZU CORPORATION, Kyoto-shi (JP)

(72) Inventors: Zhimin Xie, Kyoto-shi (JP); Tomomi TAMURA, Kyoto-shi (JP)

(21) Appl. No.: 19/454,087

(22) Filed: Jan. 20, 2026

(30) Foreign Application Priority Data
Jan. 20, 2025 (JP) 2025-007773

(51) Int. Cl. G01T 1/164; G01T 1/29

(52) U.S. Cl. CPC G01T 1/1642; G01T 1/2985

(57) ABSTRACT

A radiation imaging apparatus 100 includes: a first detector 1 that detects single gamma rays; a plurality of second detectors 2 that detect annihilation gamma rays; and an image processing unit 3 that generates a first gamma ray image 10 based on the single gamma rays detected by the first detector 1 and generates a second gamma ray image 11 based on the annihilation gamma rays detected by the plurality of second detectors 2, wherein the image processing unit 3 is configured to enhance image quality of either one of the first gamma ray image 10 and the second gamma ray image 11 using the first gamma ray image 10 and the second gamma ray image 11.

### TECHNICAL FIELD

The present invention relates to a radiation imaging apparatus and a radiation image processing method, and more particularly, to a radiation imaging apparatus and a radiation image processing method that detect gamma rays to generate a gamma ray image.

### BACKGROUND ART

Conventionally, radiation imaging apparatuses that detect gamma rays to generate a gamma ray image are known (see, for example, Patent Literature 2 and Patent Literature 1).

The partial ring PET apparatus (radiation imaging apparatus) disclosed in Patent Literature 1 includes a radiation detector for single gamma rays. The radiation detector for single gamma rays has a scattering detector and an absorption detector. The scattering detector Compton scatters single gamma rays that are emitted from an agent (radionuclide) administered to a subject and are incident on the scattering detector. The absorption detector photoelectrically absorbs scattered rays generated by Compton scattering. Thereby, a reconstructed image is generated by localizing the generation position of the gamma rays.

The nuclear medicine diagnostic apparatus (radiation imaging apparatus) described in Patent Literature 2 includes a scatterer detector, a PET (Positron Emission Tomography) detector, and an identification unit. The scatterer detector detects Compton scattering points by Compton scattering annihilation gamma rays emitted when positrons emitted from an agent (radionuclide) administered to a subject and nearby electrons undergo pair annihilation. The PET detector is configured to detect gamma rays by detecting scintillation light emitted when the annihilation gamma rays Compton scattered by the scatterer detector interact with a scintillator. The identification unit is configured to generate a gamma ray image based on the gamma rays detected by the PET detector.

### CITATION LIST

### PATENT LITERATURE

### [PATENT LITERATURE 1] JP 2018-136152 A

### [Patent Literature 2] JP 2022-061487A

### SUMMARY OF INVENTION

### TECHNICAL PROBLEM

Here, in the configuration that detects a single gamma ray (single gamma ray) as disclosed in Patent Literature 1, the radiation detector only needs to detect single gamma rays, so the number of radiation detectors can be reduced compared to the configuration that detects annihilation gamma rays as disclosed in Patent Literature 2. Furthermore, in the configuration that detects single gamma rays to generate a reconstructed image (gamma ray image), it is possible, in principle of generation, to generate a gamma ray image with a high SNR even when the number of radiation detectors is reduced. However, a gamma ray image generated by detecting single gamma rays, due to the principle of generation, suffers from streak-like (linear) artifacts, which degrades the image quality of the gamma ray image. Further, when an agent (radionuclide) with low energy of emitted single gamma rays is used in order to reduce the radiation exposure of the subject, the resolution of the generated gamma ray image decreases, and the image quality of the gamma ray image degrades.

Furthermore, in the configuration disclosed in Patent Literature 2, it is necessary to detect annihilation gamma rays, which requires multiple pairs of PET detectors. Therefore, the apparatus tends to increase in size. If the number of PET detectors is reduced to suppress the increase in the size of the apparatus, the number of annihilation gamma ray detection events by the PET detectors decreases. In this case, statistical noise is superimposed, lowering the SNR (Signal Noise Ratio), and the image quality of the generated gamma ray image degrades. Therefore, there is a demand for a radiation imaging apparatus capable of suppressing the degradation of the image quality of the generated gamma ray image even when imaging is performed under imaging conditions that cause image quality degradation, such as when using an agent (radionuclide) with low energy of emitted gamma rays or when the number of PET detectors for detecting annihilation gamma rays is reduced.

The present invention has been made to solve the problems as described above, and an object of the present invention is to provide a radiation imaging apparatus and a radiation image processing method capable of suppressing the degradation of the image quality of the generated gamma ray image even when imaging is performed under imaging conditions that cause image quality degradation, such as when using an agent (radionuclide) with low energy of emitted gamma rays or when the number of detectors for detecting annihilation gamma rays is reduced.

### SOLUTION TO PROBLEM

To achieve the above object, a radiation imaging apparatus according to a first aspect of the present invention includes: a first detector that detects single gamma rays; a plurality of second detectors that detect annihilation gamma rays; and an image processing unit that generates a first gamma ray image based on the single gamma rays detected by the first detector and generates a second gamma ray image based on the annihilation gamma rays detected by the plurality of second detectors, wherein the image processing unit is configured to enhance image quality of either one of the first gamma ray image and the second gamma ray image using the first gamma ray image and the second gamma ray image.

A radiation image processing method according to a second aspect of the present invention includes: a step of generating a first gamma ray image based on single gamma rays detected by a first detector; a step of generating a second gamma ray image based on annihilation gamma rays detected by a plurality of second detectors; and a step of enhancing image quality of either one of the first gamma ray image and the second gamma ray image using the first gamma ray image and the second gamma ray image.

### ADVANTAGEOUS EFFECTS OF INVENTION

In the radiation imaging apparatus according to the first aspect and the radiation image processing method according to the second aspect, as described above, either one of the first gamma ray image and the second gamma ray image is enhanced in image quality using the first gamma ray image generated based on single gamma rays and the second gamma ray image generated using annihilation gamma rays. Therefore, even when an agent (radionuclide) with low energy of emitted gamma rays is used, the image quality of the first gamma ray image can be enhanced using the first gamma ray image and the second gamma ray image. Further, even when the image quality of the second gamma ray image is degraded by reducing the number of second detectors, the second gamma ray image can be enhanced in image quality using the first gamma ray image and the second gamma ray image. As a result, it is possible to suppress the degradation of the image quality of the generated gamma ray image even when imaging is performed under imaging conditions that cause image quality degradation, such as when using an agent (radionuclide) with low energy of emitted gamma rays or when the number of detectors for detecting annihilation gamma rays is reduced.

### BRIEF DESCRIPTION OF DRAWINGS

FIG. 1 is a block diagram showing a configuration of a radiation imaging apparatus according to a first embodiment.

FIG. 2 is a diagram for explaining an arrangement of a first detector and second detectors according to the first embodiment.

FIG. 3 is a perspective view for explaining the arrangement of the first detector and the second detectors according to the first embodiment.

FIG. 4 is a diagram showing an example of a SPECT image.

FIG. 5 is a diagram showing an example of a PET image.

FIG. 6 is a diagram for explaining a configuration in which an image processing unit according to the first embodiment generates a first high-image-quality SPECT image.

FIG. 7 is a diagram for explaining a configuration in which the image processing unit according to the first embodiment generates a second high-image-quality SPECT image.

FIG. 8 is a flowchart for explaining processing in which the image processing unit according to the first embodiment generates the first high-image-quality SPECT image.

FIG. 9 is a flowchart for explaining processing in which the image processing unit according to the first embodiment generates the second high-image-quality SPECT image.

FIG. 10 is a block diagram showing a configuration of a radiation imaging apparatus according to a second embodiment.

FIG. 11 is a diagram for explaining a configuration in which an image processing unit according to the second embodiment generates a high-image-quality PET image.

FIG. 12 is a flowchart for explaining processing in which the image processing unit according to the second embodiment generates the high-image-quality PET image.

FIG. 13 is a block diagram showing a configuration of a radiation imaging apparatus according to a third embodiment.

### DESCRIPTION OF EMBODIMENTS

Hereinafter, an embodiment embodying the present invention will be described based on the drawings.

### First Embodiment

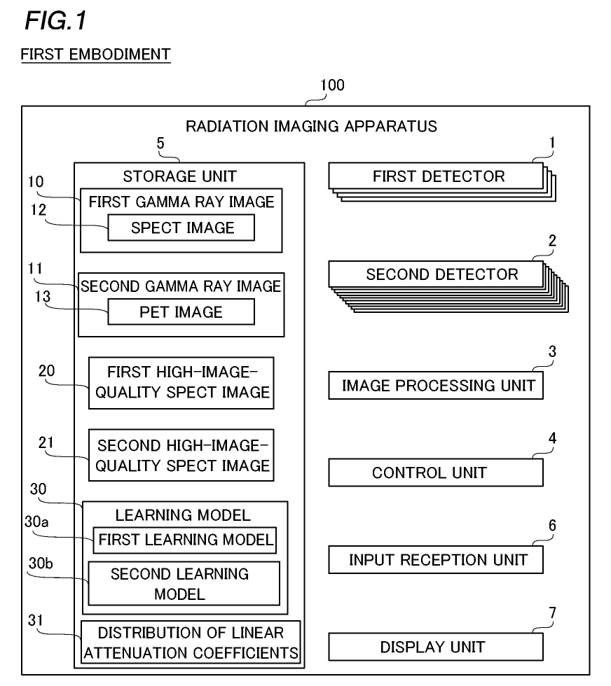

With reference to FIG. 1 to FIG. 9, a configuration of a radiation imaging apparatus 100 according to a first embodiment will be described.

### Overall Configuration of Radiation Imaging Apparatus

As shown in FIG. 1, the radiation imaging apparatus 100 includes a first detector 1, a plurality of second detectors 2, an image processing unit 3, a control unit 4, a storage unit 5, an input reception unit 6, and a display unit 7. In the first embodiment, the radiation imaging apparatus 100 includes a plurality of first detectors 1.

The radiation imaging apparatus 100 is an apparatus that administers an agent containing a radioisotope to a subject, and generates an image based on radiation (gamma rays) emitted from radionuclides contained in the agent that has accumulated in a tumor or the like within the subject's body (object 40 (see FIG. 5)). The radiation imaging apparatus 100 is configured to generate a gamma ray image used for diagnosing the subject and a gamma ray image used for confirming the therapeutic effect on the subject.

The first detector 1 is configured to detect single gamma rays. A single gamma ray is one (single) gamma ray emitted from an agent (radionuclide) administered into the subject's body. Note that the energy of single gamma rays differs depending on the agent (radionuclide) administered to the subject. In the first embodiment, the single gamma rays include first single gamma rays and second single gamma rays having different energies from each other. The first detector 1 is configured to be able to detect each of the first single gamma rays and the second single gamma rays. Details of the first detector 1 will be described later.

The plurality of second detectors 2 are configured to detect annihilation gamma rays. Details of the second detectors 2 will be described later.

Annihilation gamma rays are a pair of gamma rays emitted when a positron emitted from an agent (radionuclide) administered into the subject's body collides with a nearby electron and undergoes pair annihilation. The pair of gamma rays are emitted in directions opposite to each other (opposite directions).

The image processing unit 3 is configured to generate a first gamma ray image 10 based on the single gamma rays detected by the first detector 1. The image processing unit 3 is also configured to generate a second gamma ray image 11 based on the annihilation gamma rays detected by the plurality of second detectors 2. The image processing unit 3 includes, for example, a GPU (Graphics Processing Unit) or an FPGA (Field-Programmable Gate Array) configured for image processing.

In the first embodiment, the first gamma ray image 10 is a SPECT image 12 based on the single gamma rays detected by the first detector 1. The second gamma ray image 11 is a PET image 13 based on the annihilation gamma rays detected by the plurality of second detectors 2.

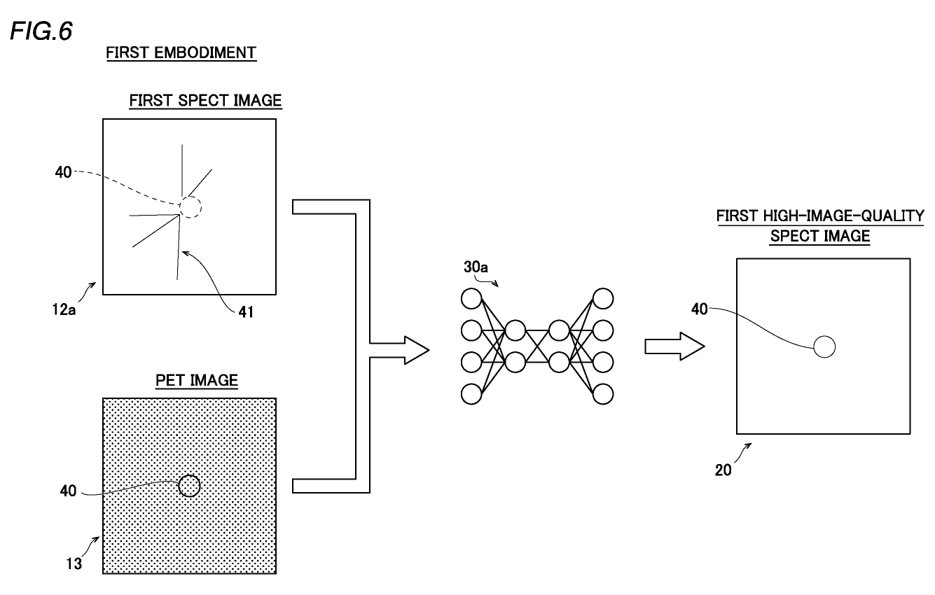

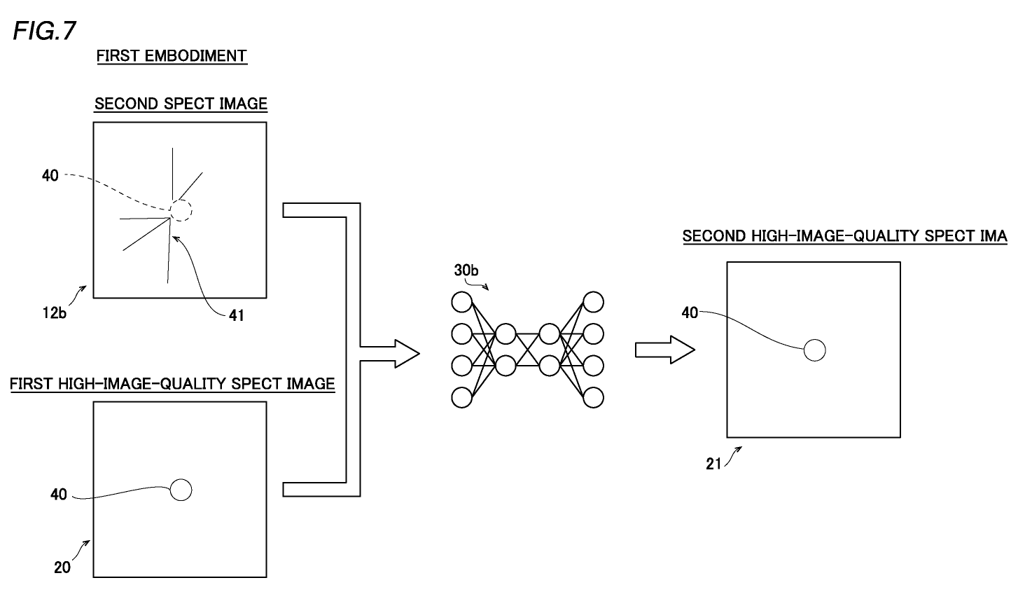

In the first embodiment, the SPECT image 12 includes a first SPECT image 12a (see FIG. 6) generated based on the first single gamma rays and a second SPECT image 12b (see FIG. 7) generated based on the second single gamma rays.

The control unit 4 is configured to control each unit of the radiation imaging apparatus 100 by executing various programs (not shown) stored in the storage unit 5. The control unit 4 includes, for example, a processor such as a CPU (Central Processing Unit) and memory such as ROM (Read Only Memory) and RAM (Random Access Memory).

The storage unit 5 stores various programs executed by the control unit 4. The storage unit 5 also stores the SPECT image 12 and the PET image 13 generated by the image processing unit 3. The storage unit 5 also stores a first high-image-quality SPECT image 20, which will be described later, and a second high-image-quality SPECT image 21, which will be described later. The storage unit 5 also stores a learning model 30, which will be described later. The storage unit 5 also stores a distribution 31 of linear attenuation coefficients, which will be described later. The storage unit 5 includes a non-volatile storage device such as an HDD (Hard Disk Drive) or an SSD (Solid State Drive).

In the first embodiment, the learning model 30 is an unsupervised deep learning model. The learning model 30 is, for example, a deep image prior network. The learning model 30 includes a first learning model 30a and a second learning model 30b. The first learning model 30a is an unsupervised deep learning model used when generating the first high-image-quality SPECT image 20, which will be described later. The second learning model 30b is an unsupervised deep learning model used when generating the second high-image-quality SPECT image 21, which will be described later.

The input reception unit 6 is configured to receive operation inputs from an operator. The input reception unit 6 includes, for example, input devices such as a mouse and a keyboard.

The display unit 7 is configured to display the SPECT image 12, the PET image 13, and the like. The display unit 7 includes, for example, a display device such as a liquid crystal monitor or an organic EL (Electro Luminescence) monitor.

### First Detector and Second Detectors

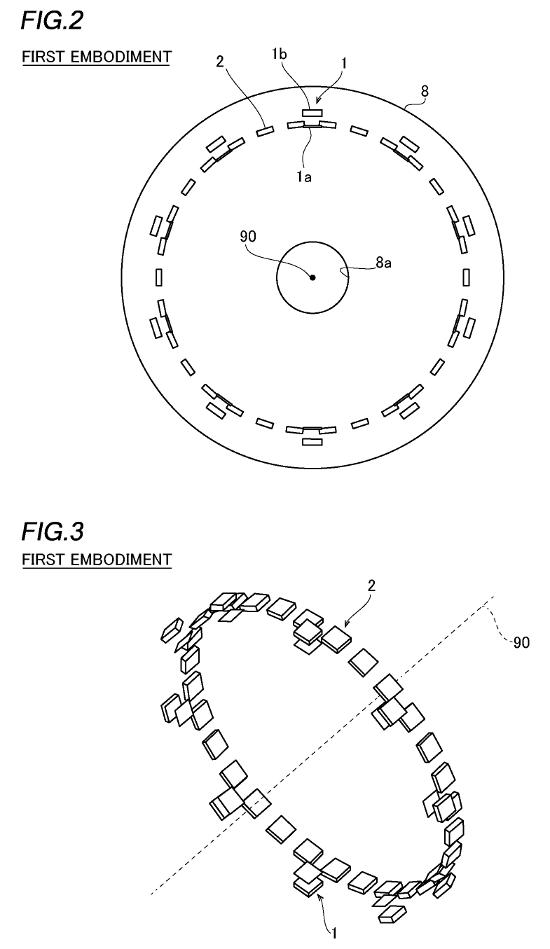

Next, with reference to FIG. 2 and FIG. 3, the configurations of the first detector 1 and the second detectors 2 will be described.

As shown in FIG. 2, the first detector 1 and the plurality of second detectors 2 are arranged inside a housing 8. The housing 8 has an opening 8a. The opening 8a is one opening and the other opening of a through hole that penetrates the housing 8 along an axis 90. When imaging a subject (object 40) with the radiation imaging apparatus 100 (see FIG. 1), the subject is placed in the through hole inside the housing 8. Note that the axis 90 is a virtual axis and does not actually exist at the center of the opening 8a.

As shown in FIG. 2, the number of first detectors 1 is smaller than the number of second detectors 2.

As shown in FIG. 2, the plurality of first detectors 1 are arranged circumferentially at predetermined intervals around the axis 90. In the example shown in FIG. 2, ten first detectors 1 are arranged circumferentially at predetermined intervals around the axis 90. Each of the plurality of first detectors 1 has a scatterer detector 1a and an absorber detector 1b.

The scatterer detector 1a is configured to Compton scatter single gamma rays emitted from the object 40. The scatterer detector 1a is also configured to detect the position (scattering point) where Compton scattering occurred. The scatterer detector 1a is, for example, a scintillator. A collimator (not shown) that restricts the direction of single gamma rays is provided on the front surface (the side toward the center of the opening 8a) of the scatterer detector 1a.

The absorber detector 1b is configured to detect scattered rays generated when single gamma rays are Compton scattered by the scatterer detector 1a. Specifically, the absorber detector 1b is configured to detect scintillation light generated by the scattered rays produced by Compton scattering. The absorber detector 1b is, for example, a photomultiplier tube.

The image processing unit 3 determines the emission position of the single gamma rays from the scattering point of Compton scattering detected by the scatterer detector 1a and the scintillation light absorbed by the absorber detector 1b, and generates the SPECT image 12 (see FIG. 1). In the first embodiment, the image processing unit 3 generates the SPECT image 12, for example, by a filtered back-projection method that back-projects projection data of single gamma rays detected by each of the plurality of first detectors 1.

The plurality of second detectors 2 are arranged circumferentially at predetermined intervals around the axis 90. In the example shown in FIG. 2, thirty second detectors 2 are arranged circumferentially at predetermined intervals around the axis 90.

Each of the plurality of second detectors 2 is configured to detect annihilation gamma rays emitted from the object 40. Each of the plurality of second detectors 2 includes a scintillator (not shown), a photodetector (not shown), and a light guide (not shown).

The scintillator converts incident annihilation gamma rays into scintillation light. The photodetector detects the scintillation light converted from the annihilation gamma rays by the scintillator. The photodetector is, for example, a photomultiplier tube. The light guide is configured to guide the scintillation light converted from the annihilation gamma rays by the scintillator to be incident on the photodetector. The light guide is composed of, for example, resin or glass, and silicon oil or the like. Note that each of the second detectors 2 may not have a light guide.

The image processing unit 3 determines the emission position of the annihilation gamma rays based on the scintillation light detected simultaneously or within a predetermined time range by the photodetectors, and generates the PET image 13 (see FIG. 2). In the first embodiment, the image processing unit 3 generates the PET image 13, for example, by an iterative image reconstruction method that compares a virtual reconstructed image with projection data of annihilation gamma rays and corrects the virtual reconstructed image so that it matches the projection data of the annihilation gamma rays.

As shown in FIG. 3, the plurality of first detectors 1 and the plurality of second detectors 2 are provided at different positions from each other in the direction along the axis 90.

### SPECT Image and PET Image

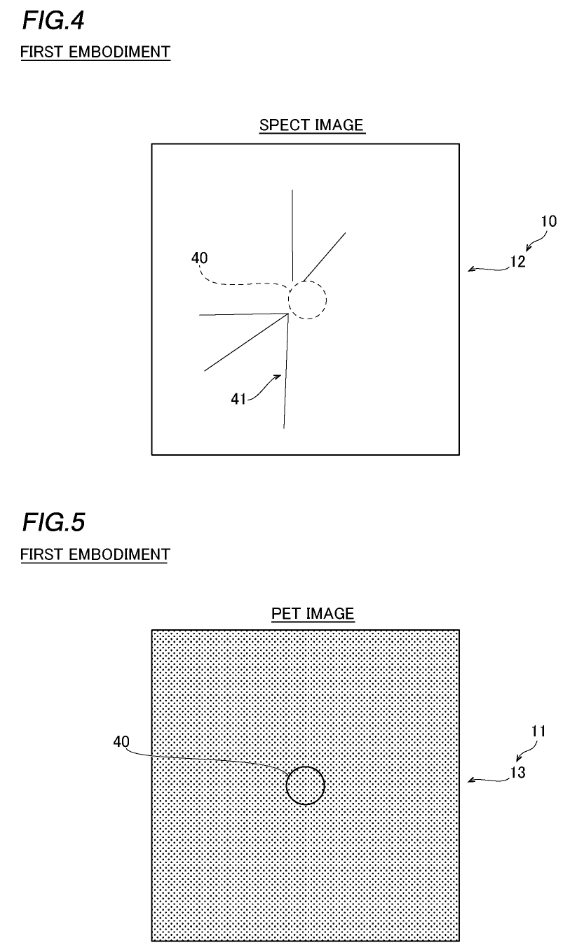

Next, with reference to FIG. 4 and FIG. 5, the SPECT image 12 and the PET image 13 will be described.

The SPECT image 12 shown in FIG. 4 is an image generated by reconstructing projection data of annihilation gamma rays detected by each of the plurality of first detectors 1 (see FIG. 2), for example, by the filtered back-projection method. When reconstructed by the filtered back-projection method, the SPECT image 12 has low resolution, and the contour of the object 40 is blurred. In the example shown in FIG. 4, the object 40 is illustrated by a dashed line to indicate that the contour of the object 40 is blurred. In the SPECT image 12, streak-like (linear) artifacts 41 may occur during reconstruction. On the other hand, statistical noise is less likely to occur in the SPECT image 12. That is, the SPECT image 12 is an image with a high SNR but low resolution of the object 40. Note that the object 40 is, for example, a tumor of the subject.

The PET image 13 shown in FIG. 5 is an image generated by reconstructing annihilation gamma rays detected by each of the plurality of second detectors 2, for example, by the iterative image reconstruction method. In the PET image 13, blurring of the contour of the object 40 and the like or streak-like artifacts 41 are less likely to occur, even if the number of the plurality of second detectors 2 is reduced. However, when the number of the plurality of second detectors 2 is reduced, statistical noise is superimposed on the PET image 13. That is, the PET image 13 is an image with high resolution of the object 40 but a low SNR. Note that in the example shown in FIG. 5, hatching is added to indicate that statistical noise is superimposed.

Therefore, in the first embodiment, the image processing unit 3 (see FIG. 1) is configured to enhance the image quality of the first gamma ray image 10 using the first gamma ray image 10 and the second gamma ray image 11. Specifically, the image processing unit 3 is configured to enhance the image quality of the first gamma ray image 10 using the first gamma ray image 10, the second gamma ray image 11, and the learning model 30 that enhances the image quality of gamma ray images.

In the first embodiment, the image processing unit 3 generates the SPECT image 12 with enhanced image quality by improving the resolution, using the SPECT image 12, the PET image 13 which contains more noise but has higher resolution than the SPECT image 12, and the learning model 30.

### Generation of First High-Image-Quality SPECT Image

The example shown in FIG. 6 shows a case where, for example, when diagnosing a subject, imaging is performed by administering to the subject an agent (radionuclide) capable of generating both the SPECT image 12 and the PET image 13. The agent (radionuclide) capable of generating both the SPECT image 12 and the PET image 13 is, for example, an agent containing 18F, from which 511 keV gamma rays are emitted.

As shown in FIG. 6, the image processing unit 3 inputs the first SPECT image 12a and the PET image 13 into the first learning model 30a. The image processing unit 3 is configured to enhance the image quality of the first gamma ray image 10 by improving the resolution of the first gamma ray image 10 by performing iterative calculations using the first learning model 30a, which is an unsupervised deep learning model, starting from a random image (not shown) to approximate the input first gamma ray image 10 (first SPECT image 12a). In the first embodiment, the image processing unit 3 is configured to generate the first high-image-quality SPECT image 20, which has enhanced image quality by improving the resolution of the SPECT image 12, using the SPECT image 12 (first SPECT image 12a) and the PET image 13.

As shown in FIG. 6, in the first high-image-quality SPECT image 20, the blurring of the contour of the object 40 is reduced compared to the first SPECT image 12a. In the first high-image-quality SPECT image 20, the streak-like artifacts 41 are suppressed compared to the first SPECT image 12a. In the first high-image-quality SPECT image 20, the superimposition of noise is suppressed compared to the PET image 13. That is, the first high-image-quality SPECT image 20 is an image with higher resolution than the first SPECT image 12a and a higher SNR than the PET image 13.

### Second High-Image-Quality SPECT Image

The example shown in FIG. 7 shows a configuration in which the image processing unit 3 enhances the image quality of the SPECT image 12 when imaging is performed to confirm the therapeutic effect on the subject. When confirming the therapeutic effect on the subject, an agent (radionuclide) different from that used during diagnosis is used. When confirming the therapeutic effect on the subject, for example, an agent containing ¹⁷⁷Lu, from which 208 keV gamma rays are emitted, is used.

In the first embodiment, the image processing unit 3 is configured to generate the second high-image-quality SPECT image 21, which has enhanced image quality by improving the resolution of the second SPECT image 12b, using the second SPECT image 12b and the first high-image-quality SPECT image 20.

In the first embodiment, the image processing unit 3 inputs the second SPECT image 12b and the first high-image-quality SPECT image 20 into the second learning model 30b. Then, the image processing unit 3 generates the second high-image-quality SPECT image 21, which has enhanced image quality by improving the resolution of the second SPECT image 12b, by performing iterative calculations starting from a random image (not shown) to approximate the input second SPECT image 12b.

As shown in FIG. 7, in the second high-image-quality SPECT image 21, the blurring of the contour of the object 40 is reduced compared to the second SPECT image 12b. In the second high-image-quality SPECT image 21, the streak-like artifacts 41 are suppressed compared to the second SPECT image 12b. That is, the second high-image-quality SPECT image 21 is an image with higher resolution than the first SPECT image 12a.

Furthermore, when treatment is performed over a long period, the therapeutic effect on the subject may be confirmed multiple times. Therefore, in the first embodiment, the image processing unit 3 generates a plurality of second SPECT images 12b based on second single gamma rays detected at different timings. Then, when enhancing the image quality of the first one of the plurality of second SPECT images 12b, the image processing unit 3 improves the resolution of the second SPECT image 12b using the first high-image-quality SPECT image 20. Furthermore, when enhancing the image quality of the second and subsequent ones of the plurality of second SPECT images 12b, the image processing unit 3 improves the resolution of the second SPECT image 12b using the second high-image-quality SPECT image 21 generated using the immediately preceding second SPECT image 12b.

### Attenuation Correction

Here, the first gamma ray image 10 and the second gamma ray image 11 are generated based on gamma rays emitted from within the subject's body (object 40). The gamma rays emitted from within the subject's body are absorbed within the subject's body. Therefore, if the gamma rays absorbed within the subject's body are not taken into account, the image quality of the generated image degrades. Therefore, in the first embodiment, the image processing unit 3 is configured to perform attenuation correction on at least one of the first gamma ray image 10 and the second gamma ray image 11 using a pre-generated distribution 31 of linear attenuation coefficients (see FIG. 1), and to enhance the image quality using the image after attenuation correction. Specifically, the image processing unit 3 is configured to perform attenuation correction on both the first gamma ray image 10 and the second gamma ray image 11. The distribution 31 of linear attenuation coefficients is created in advance by performing X-ray CT imaging on the object 40 or a phantom simulating the object 40.

### First High-Image-Quality SPECT Image Generation Processing

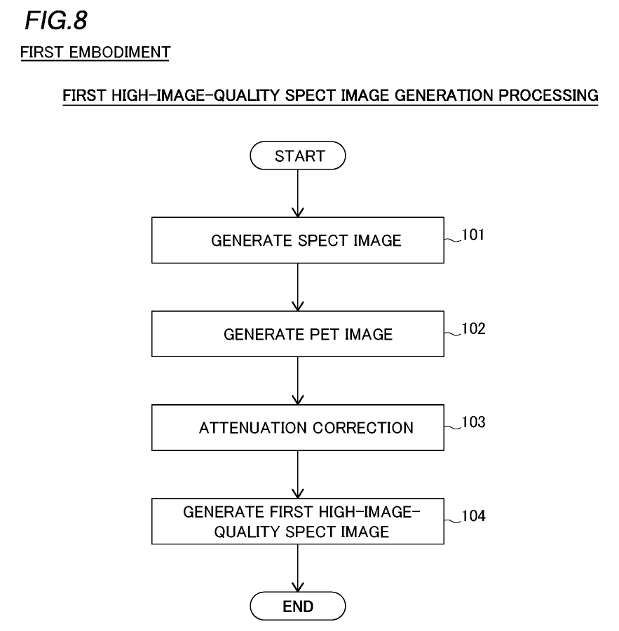

Next, with reference to FIG. 8, processing in which the image processing unit 3 (see FIG. 1) generates the first high-image-quality SPECT image 20 (see FIG. 6) will be described.

In step 101, the image processing unit 3 generates the first gamma ray image 10 based on the single gamma rays detected by the plurality of first detectors 1. Specifically, the image processing unit 3 generates the first SPECT image 12a (see FIG. 6) based on the single gamma rays detected by the plurality of first detectors 1.

Next, in step 102, the image processing unit 3 generates the second gamma ray image 11 based on the annihilation gamma rays detected by the plurality of second detectors 2. In the first embodiment, the image processing unit 3 generates the PET image 13 (see FIG. 6) based on the annihilation gamma rays detected by the plurality of second detectors 2.

Next, in step 103, the image processing unit 3 performs attenuation correction on each of the first SPECT image 12a and the PET image 13. In the first embodiment, the image processing unit 3 performs attenuation correction on each of the first SPECT image 12a and the PET image 13 using the distribution 31 of linear attenuation coefficients, and re-outputs each image.

Next, in step 104, the image processing unit 3 enhances the image quality of the first gamma ray image 10 using the first gamma ray image 10 and the second gamma ray image 11. In the first embodiment, the image processing unit 3 generates the first high-image-quality SPECT image 20, which has enhanced image quality of the first SPECT image 12a, by inputting the first SPECT image 12a after attenuation correction and the PET image 13 after attenuation correction into the first learning model 30a. Thereafter, the processing ends.

Note that the processing of step 101 and the processing of step 102 may be performed in either order.

### Second High-Image-Quality SPECT Image Generation Processing

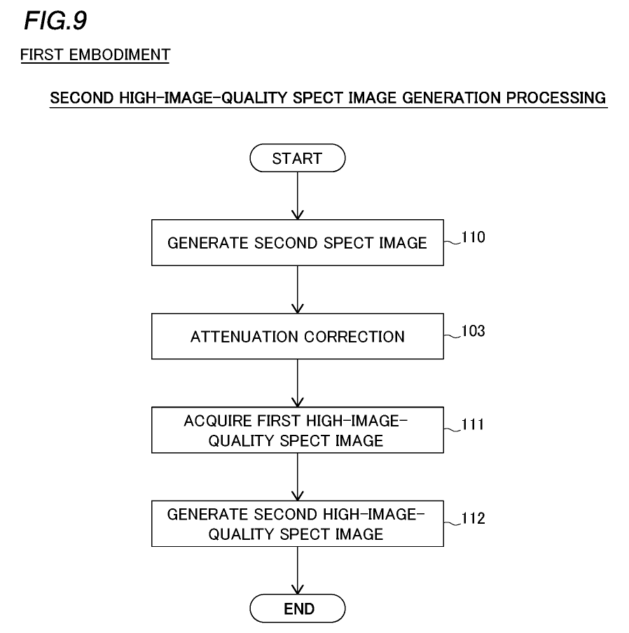

Next, with reference to FIG. 9, processing in which the image processing unit 3 (see FIG. 1) generates the second high-image-quality SPECT image 21 (see FIG. 7) will be described. Note that processing similar to the processing for generating the first high-image-quality SPECT image 20 described above is denoted by the same reference numerals, and detailed description thereof is omitted.

In step 110, the image processing unit 3 generates the second SPECT image 12b. Specifically, the image processing unit 3 generates the second high-image-quality SPECT image 21 based on the second single gamma rays.

Next, in step 103, the image processing unit 3 performs attenuation correction on the second SPECT image 12b.

Next, in step 111, the image processing unit 3 acquires the first high-image-quality SPECT image 20. In the first embodiment, the image processing unit 3 acquires the first high-image-quality SPECT image 20, which was generated in advance during diagnosis of the subject or the like and stored in the storage unit 5 (see FIG. 1), from the storage unit 5.

Next, in step 112, the image processing unit 3 generates the second high-image-quality SPECT image 21. Specifically, the image processing unit 3 generates the second high-image-quality SPECT image 21 by inputting the second SPECT image 12b and the first high-image-quality SPECT image 20 into the second learning model 30b (see FIG. 7) and performing iterative calculations. Thereafter, the processing ends.

Note that the processing of step 110 and step 103, and the processing of step 111 may be performed in either order.

### Second Embodiment

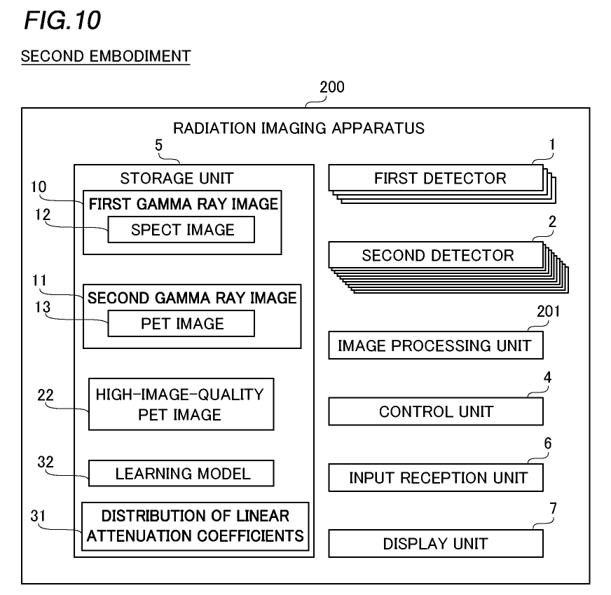

Next, with reference to FIG. 10 to FIG. 12, a second embodiment will be described. In a radiation imaging apparatus 200 according to the second embodiment, a configuration in which an image processing unit 201 enhances the image quality of the PET image 13 will be described. Note that components similar to those in the first embodiment described above are denoted by the same reference numerals, and detailed description thereof is omitted.

### Overall Configuration of Radiation Imaging Apparatus

As shown in FIG. 10, the radiation imaging apparatus 200 according to the second embodiment includes the plurality of first detectors 1, the plurality of second detectors 2, the control unit 4, the storage unit 5, the input reception unit 6, the display unit 7, and an image processing unit 201.

In the second embodiment, the storage unit 5 stores the first gamma ray image 10 (SPECT image 12), the second gamma ray image 11 (PET image 13), a high-image-quality PET image 22, which will be described later, the distribution 31 of linear attenuation coefficients, and a learning model 32 used when generating the high-image-quality PET image 22.

In the second embodiment, the image processing unit 201 is configured to enhance the image quality of the second gamma ray image 11 using the first gamma ray image 10 and the second gamma ray image 11. Specifically, the image processing unit 201 is configured to enhance the image quality of the second gamma ray image 11 using the first gamma ray image 10, the second gamma ray image 11, and the learning model 32 that enhances the image quality of gamma ray images. More specifically, the image processing unit 201 generates the PET image 13 with enhanced image quality by reducing noise, using the SPECT image 12, the PET image 13, and the learning model 30.

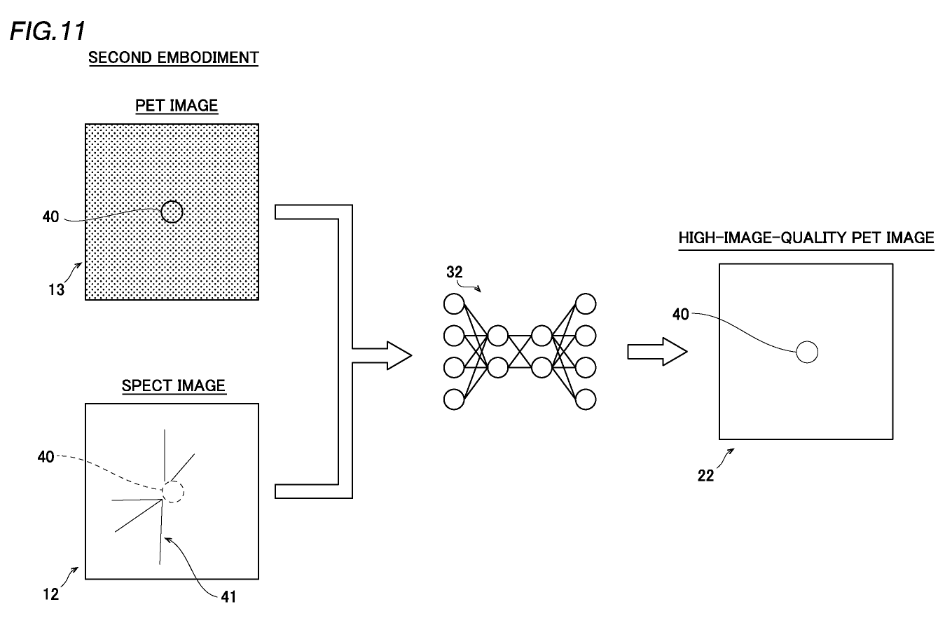

As shown in FIG. 11, the image processing unit 201 (see FIG. 10) according to the second embodiment is configured to enhance the image quality of the second gamma ray image 11 by reducing the noise of the second gamma ray image 11 by performing iterative calculations using the learning model 32, which is an unsupervised deep learning model, starting from a random image to approximate the input second gamma ray image 11 (PET image 13). Specifically, the image processing unit 201 is configured to generate the high-image-quality PET image 22, which has enhanced image quality by reducing the noise of the PET image 13, using the SPECT image 12 and the PET image 13.

As shown in FIG. 11, in the high-image-quality PET image 22, the superimposition of noise is suppressed compared to the PET image 13. In the high-image-quality PET image 22, the blurring of the contour of the object 40 is reduced compared to the SPECT image 12. In the high-image-quality PET image 22, the streak-like artifacts 41 are suppressed compared to the SPECT image 12. That is, the high-image-quality PET image 22 is an image with a higher SNR than the PET image 13 and higher resolution than the SPECT image 12.

### High-Image-Quality PET Image Generation Processing

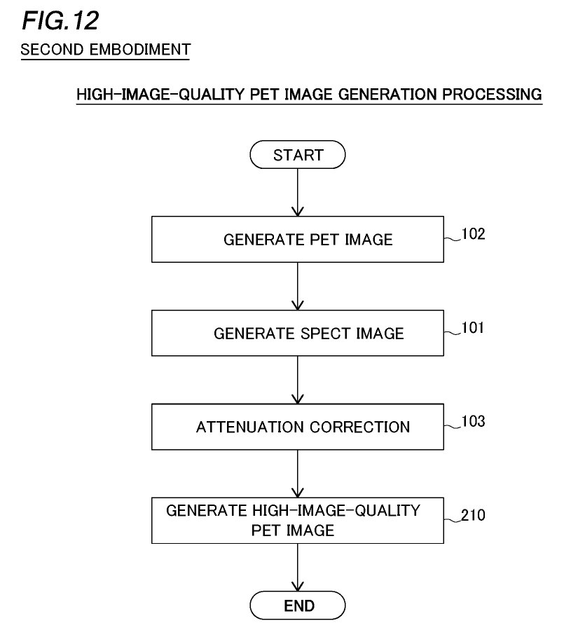

Next, with reference to FIG. 12, processing in which the image processing unit 201 (see FIG. 10) generates the high-image-quality PET image 22 (see FIG. 10) will be described. Note that processing similar to the processing in which the image processing unit 3 (see FIG. 1) according to the first embodiment generates the first high-image-quality SPECT image 20 (see FIG. 7) is denoted by the same reference numerals, and detailed description thereof is omitted.

In steps 102 and 101, the image processing unit 201 generates the SPECT image 12 and the PET image 13. In step 103, the image processing unit 201 performs attenuation correction on each of the SPECT image 12 and the PET image 13. Note that the processing of step 102 and the processing of step 101 may be performed in either order.

Next, in step 210, the image processing unit 201 generates the high-image-quality PET image 22. Specifically, the image processing unit 201 generates the high-image-quality PET image 22 by inputting the PET image 13 after attenuation correction and the SPECT image 12 after attenuation correction into the learning model 32. Thereafter, the processing ends.

The other configurations of the second embodiment are the same as the configurations of the first embodiment.

### Third Embodiment

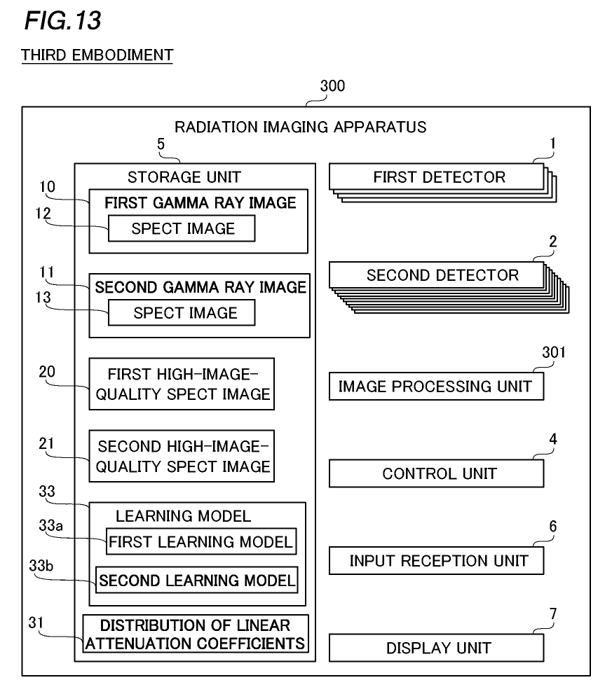

Next, with reference to FIG. 13, a third embodiment will be described. In a radiation imaging apparatus 300 according to the third embodiment, a configuration in which an image processing unit 301 enhances the image quality of the SPECT image 12 using a learning model 33 will be described. Note that components similar to those in the first embodiment described above are denoted by the same reference numerals, and detailed description thereof is omitted.

### Overall Configuration of Radiation Imaging Apparatus

As shown in FIG. 13, the radiation imaging apparatus 300 according to the third embodiment includes the plurality of first detectors 1, the plurality of second detectors 2, the control unit 4, the storage unit 5, the input reception unit 6, the display unit 7, and an image processing unit 301.

In the third embodiment, the storage unit 5 stores the first gamma ray image 10 (SPECT image 12), the second gamma ray image 11 (PET image 13), the first high-image-quality SPECT image 20, the second high-image-quality SPECT image 21, the distribution 31 of linear attenuation coefficients, and a learning model 33 used when generating the first high-image-quality SPECT image 20 and the second high-image-quality SPECT image 21.

The learning model 33 is a supervised deep learning model. The learning model 33 includes, for example, a Convolutional Neural Network (CNN) or a U-net.

The learning model 33 is generated by learning to enhance the image quality of the SPECT image 12 using training data.

The learning model 33 includes a first learning model 33a and a second learning model 33b.

The first learning model 33a is generated by learning to enhance the image quality of the SPECT image 12 (first SPECT image 12a) using the SPECT image 12 (first SPECT image 12a) and the PET image 13. The second learning model 33b is generated by learning to enhance the image quality of the SPECT image 12 (second SPECT image 12b) using the SPECT image 12 (second SPECT image 12b) and the first high-image-quality SPECT image 20.

In the third embodiment, the image processing unit 301 is configured to enhance the image quality of the first gamma ray image 10 by improving the resolution of the first gamma ray image 10, using the learning model 33 that has learned to improve the resolution of the first gamma ray image 10.

Specifically, the image processing unit 301 generates the first high-image-quality SPECT image 20, which has enhanced image quality of the first SPECT image 12a, using the first SPECT image 12a, the PET image 13, and the first learning model 33a.

The image processing unit 301 also generates the second high-image-quality SPECT image 21, which has enhanced image quality of the second SPECT image 12b, using the second SPECT image 12b, the first high-image-quality SPECT image 20, and the second learning model 33b.

The other configurations of the third embodiment are the same as the configurations of the first embodiment.

### MODIFICATIONS

It should be understood that the embodiments disclosed this time are illustrative in all respects and not restrictive. The scope of the present invention is indicated by the claims rather than by the description of the embodiments described above, and all changes (modifications) within the meaning and scope equivalent to the claims are intended to be included.

For example, the image processing unit may enhance the image quality of the first gamma ray image (SPECT image) or the second gamma ray image (PET image) without using a learning model. In this case, the image processing unit may enhance the image quality of the SPECT image or the PET image by performing a weighted average of the SPECT image and the PET image. Furthermore, the number of first detectors is merely an example, and may be a number other than the number shown in the first embodiment. The number of first detectors may be one. In this case, the first detector may be configured to detect single gamma rays while rotating around the axis passing through the center of the opening. Furthermore, the number of second detectors is merely an example, and may be a plurality of any number other than the number shown in the first embodiment. Furthermore, the image processing unit only needs to generate at least the first high-image-quality SPECT image, and does not necessarily have to generate the second high-image-quality SPECT image. Furthermore, when generating the second high-image-quality SPECT image, the image processing unit may use the second high-image-quality SPECT image generated using the second SPECT image from two images prior. Furthermore, the image processing unit may perform attenuation correction during the reconstruction of the first gamma ray image and the reconstruction of the second gamma ray image. Furthermore, the image processing unit may perform scatter correction or the like together with attenuation correction. Furthermore, the image processing unit does not necessarily have to perform attenuation correction. Furthermore, the image processing unit may enhance the image quality of the second gamma ray image (PET image) by reducing the noise of the second gamma ray image, using a learning model that is a supervised deep learning model that has learned to reduce the noise of the second gamma ray image. Furthermore, a configuration combining the first embodiment and the second embodiment may be adopted. That is, the image processing unit may be configured to be able to enhance the image quality of the first gamma ray image and enhance the image quality of the second gamma ray image, using the first gamma ray image and the second gamma ray image. Furthermore, although each processing performed by the image processing unit has been described using a flow-driven flowchart that performs processing sequentially according to the processing flow, the present invention is not limited to this. In the present invention, each processing performed by the image processing unit may be performed by event-driven processing that executes processing in units of events. In this case, it may be performed by a completely event-driven method, or by a combination of event-driven and flow-driven methods.

### Aspects

It is understood by those skilled in the art that the exemplary embodiments described above are specific examples of the following aspects.

(Item 1) A radiation imaging apparatus, comprising: a first detector that detects single gamma rays; a plurality of second detectors that detect annihilation gamma rays; and an image processing unit that generates a first gamma ray image based on the single gamma rays detected by the first detector and generates a second gamma ray image based on the annihilation gamma rays detected by the plurality of second detectors, wherein the image processing unit is configured to enhance image quality of either one of the first gamma ray image and the second gamma ray image using the first gamma ray image and the second gamma ray image. Either one of the first gamma ray image and the second gamma ray image is enhanced in image quality using the first gamma ray image generated based on single gamma rays and the second gamma ray image generated using annihilation gamma rays. Therefore, even when an agent (radionuclide) with low energy of emitted gamma rays is used, the image quality of the first gamma ray image can be enhanced using the first gamma ray image and the second gamma ray image. Further, even when the image quality of the second gamma ray image is degraded by reducing the number of second detectors, the second gamma ray image can be enhanced in image quality using the first gamma ray image and the second gamma ray image. As a result, it is possible to suppress the degradation of the image quality of the generated gamma ray image even when imaging is performed under imaging conditions that cause image quality degradation, such as when using an agent (radionuclide) with low energy of emitted gamma rays or when the number of detectors (second detectors) for detecting annihilation gamma rays is reduced.

(Item 2) The radiation imaging apparatus according to item 1, wherein the image processing unit is configured to enhance the image quality of either one of the first gamma ray image and the second gamma ray image using the first gamma ray image, the second gamma ray image, and a learning model that enhances image quality of gamma ray images. Since it becomes possible to enhance the image quality of the first gamma ray image or the second gamma ray image using the learning model, the first gamma ray image or the second gamma ray image can be easily enhanced in image quality compared to a configuration in which the image quality of the first gamma ray image or the second gamma ray image is enhanced by image processing without using a learning model. As a result, it is possible to provide a radiation imaging apparatus capable of easily enhancing the image quality of the first gamma ray image or the second gamma ray image even when the image quality of the first gamma ray image and the second gamma ray image is low.

(Item 3) The radiation imaging apparatus according to item 2, wherein the first gamma ray image is a SPECT image based on the single gamma rays detected by the first detector, the second gamma ray image is a PET image based on the annihilation gamma rays detected by the plurality of second detectors, and the image processing unit generates, using the SPECT image, the PET image which contains more noise but has higher resolution than the SPECT image, and the learning model, the SPECT image with enhanced image quality by improving resolution, or the PET image with enhanced image quality by reducing noise. Even when an agent (radionuclide) with low energy of emitted gamma rays is used, a SPECT image with improved resolution can be generated. Further, even when the number of the plurality of second detectors is reduced, a PET image with reduced noise can be generated. As a result, even when imaging is performed under imaging conditions that cause image quality degradation, such as when using an agent (radionuclide) with low energy of emitted gamma rays or when the number of detectors (second detectors) for detecting annihilation gamma rays is reduced, it is possible to generate the SPECT image with enhanced image quality by improving resolution, or the PET image with enhanced image quality by reducing noise.

(Item 4) The radiation imaging apparatus according to item 3, wherein the image processing unit is configured to generate a first high-image-quality SPECT image, which has enhanced image quality by improving resolution of the SPECT image, using the SPECT image and the PET image. Even when an agent (radionuclide) with low energy of emitted gamma rays is used, the first high-image-quality SPECT image with improved resolution can be generated. As a result, it is possible to provide a radiation imaging apparatus capable of generating the first high-image-quality SPECT image with enhanced image quality by improving resolution, even when an agent (radionuclide) with low energy of emitted gamma rays is used. Further, since it becomes possible to generate the first high-image-quality SPECT image with improved resolution even when an agent (radionuclide) with low energy of emitted gamma rays is used, it is possible to generate the first high-image-quality SPECT image that allows a doctor or the like to accurately grasp the position and size of a tumor. As a result, the burden on the subject can be reduced.

(Item 5) The radiation imaging apparatus according to item 4, wherein the single gamma rays include first single gamma rays and second single gamma rays having different energies from each other, the first detector is configured to be able to detect each of the first single gamma rays and the second single gamma rays, the SPECT image includes a first SPECT image generated based on the first single gamma rays and a second SPECT image generated based on the second single gamma rays, and the image processing unit is configured to generate a second high-image-quality SPECT image, which has enhanced image quality by improving resolution of the second SPECT image, using the second SPECT image and the first high-image-quality SPECT image. Here, the agent (radionuclide) used for imaging when diagnosing a subject and the agent (radionuclide) used for imaging when confirming the therapeutic effect on the subject may be different from each other. In that case, the energy of the first single gamma rays and the energy of the second single gamma rays will be different energies from each other. For example, during diagnosis, if imaging is performed using an agent (radionuclide) that emits single gamma rays and annihilation gamma rays with energies detectable by both the first detector and the second detector, the first SPECT image generated using the first single gamma rays can be enhanced in image quality using the PET image. On the other hand, when confirming the therapeutic effect on the subject, an agent (radionuclide) that emits gamma rays with an energy that allows a SPECT image to be generated but does not allow a PET image to be generated may be used. In this case, the second SPECT image generated based on the second single gamma rays cannot be enhanced in image quality using the PET image. Therefore, by adopting the configuration as described above, by enhancing the image quality of the second SPECT image using the first high-image-quality SPECT image, which has been enhanced in image quality using the first SPECT image generated during diagnosis and the PET image, it is possible to generate the second high-image-quality SPECT image, which has enhanced image quality of the second SPECT image, even when the energy of the gamma rays emitted from the agent (radionuclide) used when confirming the therapeutic effect on the subject is an energy that does not allow a PET image to be generated.

(Item 6) The radiation imaging apparatus according to item 5, wherein the image processing unit generates a plurality of the second SPECT images based on the second single gamma rays detected at different timings, improves the resolution of the second SPECT image using the first high-image-quality SPECT image when enhancing the image quality of a first one of the plurality of second SPECT images, and improves the resolution of the second SPECT image using the second high-image-quality SPECT image generated using an immediately preceding one of the second SPECT images when enhancing the image quality of a second or subsequent one of the plurality of second SPECT images. Here, when radiation imaging is repeatedly performed to confirm the therapeutic effect on the subject, the object may have changed between the not-latest (two or more images prior) second high-image-quality SPECT image and the latest second SPECT image. In this case, even if an attempt is made to improve the resolution of the latest second SPECT image using the not-latest second high-image-quality SPECT image, the resolution of the second SPECT image may not be improved due to the difference in the object. Therefore, as described above, by improving the resolution of the latest second SPECT image using the latest (immediately preceding) second high-image-quality SPECT image when enhancing the image quality of the second or subsequent second SPECT images, it is possible to suppress the improvement of the resolution of the latest second SPECT image using the not-latest (two or more images prior) second high-image-quality SPECT image. As a result, the resolution of the latest second SPECT image can be improved more accurately.

(Item 7) The radiation imaging apparatus according to item 3, wherein the image processing unit is configured to generate a high-image-quality PET image, which has enhanced image quality by reducing noise of the PET image, using the SPECT image and the PET image. Even when the number of the plurality of second detectors is reduced, the high-image-quality PET image with reduced noise can be generated. As a result, it is possible to provide a radiation imaging apparatus capable of generating the high-image-quality PET image with reduced noise even when the number of second detectors for detecting annihilation gamma rays is reduced.

(Item 8) The radiation imaging apparatus according to item 2 or 3, wherein the learning model is an unsupervised deep learning model, and the image processing unit is configured to enhance the image quality of the first gamma ray image by improving the resolution of the first gamma ray image, or to enhance the image quality of the second gamma ray image by reducing the noise of the second gamma ray image, by performing iterative calculations using the learning model, which is an unsupervised deep learning model, starting from a random image to approximate the input first gamma ray image or second gamma ray image. When the learning model is an unsupervised deep learning model, since iterative calculations are performed starting from a random image to approximate the first gamma ray image or the second gamma ray image, the image quality of the first gamma ray image or the second gamma ray image can be enhanced regardless of the part of the object captured in the first gamma ray image or the second gamma ray image. As a result, it becomes possible to enhance the image quality of the first gamma ray image or the second gamma ray image using a highly versatile learning model, so the user's burden required to generate the learning model can be reduced compared to a configuration in which a dedicated learning model is generated for each part of the object to enhance the image quality of the first gamma ray image or the second gamma ray image.

(Item 9) The radiation imaging apparatus according to item 2 or 3, wherein the learning model is a supervised deep learning model, and the image processing unit is configured to enhance the image quality of the first gamma ray image by improving the resolution of the first gamma ray image, or to enhance the image quality of the second gamma ray image by reducing the noise of the second gamma ray image, using the learning model that has learned to improve the resolution of the first gamma ray image or to reduce the noise of the second gamma ray image. When the learning model is a supervised deep learning model, since the learning model is generated by learning according to the part of the object captured in the first gamma ray image or the second gamma ray image, the image quality of the first gamma ray image or the second gamma ray image can be enhanced using the learning model specialized for the part of the object. As a result, the image quality of the first gamma ray image or the second gamma ray image can be further enhanced.

(Item 10) The radiation imaging apparatus according to any one of items 1 to 9, wherein the image processing unit is configured to perform attenuation correction on at least one of the first gamma ray image and the second gamma ray image using a pre-generated distribution of linear attenuation coefficients, and to enhance the image quality using the image after the attenuation correction. Here, the gamma rays emitted from within the subject's body (object) are absorbed by the subject's body. The degree to which gamma rays are absorbed becomes non-uniform according to the difference in the distance from the position where the gamma rays are emitted to the body surface. Therefore, by performing attenuation correction on at least one of the first gamma ray image or the second gamma ray image using the distribution of linear attenuation coefficients, it becomes possible to eliminate the non-uniformity of gamma ray absorption, and the distribution of gamma rays emitted from within the subject's body can be accurately acquired. As a result, the first gamma ray image or the second gamma ray image before image quality enhancement can be generated accurately, so the image quality of the first gamma ray image or the second gamma ray image can be further enhanced.

(Item 11) A radiation image processing method, comprising: a step of generating a first gamma ray image based on single gamma rays detected by a first detector; a step of generating a second gamma ray image based on annihilation gamma rays detected by a plurality of second detectors; and a step of enhancing image quality of either one of the first gamma ray image and the second gamma ray image using the first gamma ray image and the second gamma ray image. Similarly to the radiation imaging apparatus, it is possible to provide a radiation image processing method capable of suppressing the degradation of the image quality of the generated gamma ray image even when imaging is performed under imaging conditions that cause image quality degradation, such as when using an agent (radionuclide) with low energy of emitted gamma rays or when the number of detectors (second detectors) for detecting annihilation gamma rays is reduced.

### REFERENCE SIGNS LIST

1 First detector 2 Second detector 3, 201, 301 Image processing unit 10 First gamma ray image 11 Second gamma ray image 12 SPECT image 12a First SPECT image 12b Second SPECT image 13 PET image 20 First high-image-quality SPECT image 21 Second high-image-quality SPECT image 22 High-image-quality PET image 30, 32, 33 Learning model 31 Distribution of linear attenuation coefficients 100, 200, 300 Radiation imaging apparatus

1. A radiation imaging apparatus, comprising: a first detector that detects single gamma rays; a plurality of second detectors that detect annihilation gamma rays; and an image processing unit that generates a first gamma ray image based on the single gamma rays detected by the first detector and generates a second gamma ray image based on the annihilation gamma rays detected by the plurality of second detectors, wherein the image processing unit is configured to enhance image quality of either one of the first gamma ray image and the second gamma ray image using the first gamma ray image and the second gamma ray image.

2. The radiation imaging apparatus according to claim 1, wherein the image processing unit is configured to enhance the image quality of either one of the first gamma ray image and the second gamma ray image using the first gamma ray image, the second gamma ray image, and a learning model that enhances image quality of gamma ray images.

3. The radiation imaging apparatus according to claim 2, wherein the first gamma ray image is a SPECT image based on the single gamma rays detected by the first detector, the second gamma ray image is a PET image based on the annihilation gamma rays detected by the plurality of second detectors, and the image processing unit generates, using the SPECT image, the PET image which contains more noise but has higher resolution than the SPECT image, and the learning model, the SPECT image with enhanced image quality by improving resolution, or the PET image with enhanced image quality by reducing noise.

4. The radiation imaging apparatus according to claim 3, wherein the image processing unit is configured to generate a first high-image-quality SPECT image, which has enhanced image quality by improving resolution of the SPECT image, using the SPECT image and the PET image.

5. The radiation imaging apparatus according to claim 4, wherein the single gamma rays include first single gamma rays and second single gamma rays having different energies from each other, the first detector is configured to be able to detect each of the first single gamma rays and the second single gamma rays, the SPECT image includes a first SPECT image generated based on the first single gamma rays and a second SPECT image generated based on the second single gamma rays, and the image processing unit is configured to generate a second high-image-quality SPECT image, which has enhanced image quality by improving resolution of the second SPECT image, using the second SPECT image and the first high-image-quality SPECT image.

6. The radiation imaging apparatus according to claim 5, wherein the image processing unit generates a plurality of the second SPECT images based on the second single gamma rays detected at different timings, improves the resolution of the second SPECT image using the first high-image-quality SPECT image when enhancing the image quality of a first one of the plurality of second SPECT images, and improves the resolution of the second SPECT image using the second high-image-quality SPECT image generated using an immediately preceding one of the second SPECT images when enhancing the image quality of a second or subsequent one of the plurality of second SPECT images.

7. The radiation imaging apparatus according to claim 3, wherein the image processing unit is configured to generate a high-image-quality PET image, which has enhanced image quality by reducing noise of the PET image, using the SPECT image and the PET image.

8. The radiation imaging apparatus according to claim 3, wherein the learning model is an unsupervised deep learning model, and the image processing unit is configured to enhance the image quality of the first gamma ray image by improving the resolution of the first gamma ray image, or to enhance the image quality of the second gamma ray image by reducing the noise of the second gamma ray image, by performing iterative calculations using the learning model, which is an unsupervised deep learning model, starting from a random image to approximate the input first gamma ray image or second gamma ray image.

9. The radiation imaging apparatus according to claim 3, wherein the learning model is a supervised deep learning model, and the image processing unit is configured to enhance the image quality of the first gamma ray image by improving the resolution of the first gamma ray image, or to enhance the image quality of the second gamma ray image by reducing the noise of the second gamma ray image, using the learning model that has learned to improve the resolution of the first gamma ray image or to reduce the noise of the second gamma ray image.

10. The radiation imaging apparatus according to claim 1, wherein the image processing unit is configured to perform attenuation correction on at least one of the first gamma ray image and the second gamma ray image using a pre-generated distribution of linear attenuation coefficients, and to enhance the image quality using the image after the attenuation correction.

11. A radiation image processing method, comprising: a step of generating a first gamma ray image based on single gamma rays detected by a first detector; a step of generating a second gamma ray image based on annihilation gamma rays detected by a plurality of second detectors; and a step of enhancing image quality of either one of the first gamma ray image and the second gamma ray image using the first gamma ray image and the second gamma ray image.
